(19) United States
(12) Patent Application Publication
(10) Pub. No.: US 2023/0226251 A1
(43) Pub. Date: Jul. 20, 2023

(54) WOUND CARE COVERING

(71) Applicant: Boock Engineering LLC, Carlsbad, CA (US)

(72) Inventor: Robert James Boock, Carlsbad, CA (US)

(73) Assignee: Boock Engineering LLC, Carlsbad, CA (US)

(21) Appl. No.: 18/062,736

(22) Filed: Dec. 7, 2022

Related U.S. Application Data
(60) Provisional application No. 63/266,847, filed on Jan. 17, 2022.

(51) Int. Cl. A61L 15/44; A61L 15/38; A61L 15/28; A61L 15/26; A61L 15/56

(52) U.S. Cl. CPC A61L 15/44; A61L 15/38; A61L 15/28; A61L 15/26; A61L 15/56; A61L 2300/442; A61L 2300/61; A61L 2300/254

(57) ABSTRACT

Wound care coverings include a pad comprising a pad material, a lactate-based enzyme crosslinked with the pad material by a crosslinker, and a dye that changes color when H₂O₂ is present. In examples, an enzyme layer comprises a pad material crosslinked with a sugar-based enzyme by a crosslinker, a sugar-containing layer comprises a sugar in a hydrogel, and a rupturable barrier is between the enzyme layer and sugar-containing layer. In examples, a first enzyme layer comprises a first pad material, a lactate-based enzyme and a dye that changes color when H₂O₂ is present; a second enzyme layer comprises a second pad material crosslinked with a sugar-based enzyme by a second crosslinker; a sugar-containing layer comprises a sugar in a hydrogel, where the second enzyme layer is between the sugar-containing layer and the first enzyme layer; and a rupturable barrier is between the second enzyme layer and the sugar-containing layer.

### RELATED APPLICATIONS

This application claims priority to U.S. Provisional Patent Application No. 63/266,847, filed on Jan. 17, 2022, and entitled “Wound Care Covering,” which is hereby incorporated by reference for all purposes.

### BACKGROUND

Wound care coverings such as adhesive bandages are important for the healing of skin wounds, by protecting the injured area from contaminants and thus helping to prevent infection. Adhesive bandages and wound dressings come in various forms, such as having waterproofing, different backing materials to provide durability during usage, and pad materials that prevent sticking to the wound. Some bandages offer even further defensive properties by incorporating an antibiotic substance in the pad of the bandage.

Adhesive bandages are used ubiquitously in both home and professional care settings and are essential as a first line of defense in the prevention of more serious medical issues in wounds.

### SUMMARY

In embodiments, a wound care covering includes a pad having a pad material. A lactate-based enzyme is crosslinked with the pad material by a crosslinker. The wound care covering also includes a dye that changes color when hydrogen peroxide is present.

In embodiments, a wound care covering includes an enzyme layer comprising a pad material crosslinked with a sugar-based enzyme by a crosslinker. A sugar-containing layer includes a sugar in a hydrogel. A rupturable barrier is between the enzyme layer and the sugar-containing layer.

In embodiments, a wound care covering includes a first enzyme layer comprising a first pad material, a lactate-based enzyme, and a dye that changes color when hydrogen peroxide is present, wherein the lactate-based enzyme is crosslinked with the first pad material by a first crosslinker. A second enzyme layer comprises a second pad material crosslinked with a sugar-based enzyme by a second crosslinker. A sugar-containing layer comprises a sugar in a hydrogel, wherein the second enzyme layer is between the sugar-containing layer and the first enzyme layer. A rupturable barrier is between the second enzyme layer and the sugar-containing layer.

### BRIEF DESCRIPTION OF THE DRAWINGS

FIGS. 1A-1C show views of a wound care covering that indicates the presence of an infection, in accordance with some embodiments.

FIGS. 2A-2C show views of a wound care covering that helps prevent development of an infection, in accordance with some embodiments.

FIGS. 3A-3B show views of a wound care covering that helps prevent an infection from developing and also indicates the presence of an infection, in accordance with some embodiments.

FIG. 4 is a flowchart of methods for making a wound care covering, in accordance with some embodiments.

FIG. 5 shows an image of an example pad for a wound care covering, in accordance with some embodiments.

FIG. 6 is a graph of experimental results for hydrogen peroxide generated by a pad in response to glucose, in accordance with some embodiments.

### DETAILED DESCRIPTION

Wound care coverings are disclosed that provide diagnostic and/or therapeutic capabilities for infections in skin wounds. In some embodiments, wound care coverings have a pad that indicates the presence of an infection by detecting a rise in lactate levels in the wound. Such wound care coverings can provide real-time monitoring for the development of an infection, so that the infection can be treated promptly. In some embodiments, wound care coverings have a pad that helps prevent infection by producing hydrogen peroxide at levels that promote both antiseptic and wound healing activity rather than impairing tissue growth. Such wound care coverings can reduce or eliminate the need for a user to apply other products (e.g., antibiotics, antiseptics, rubbing alcohol) manually, and also enable infection-prevention substances to be applied on an ongoing basis to a wound. Some embodiments include wound care coverings that have both the infection detection and prevention capabilities.

Embodiments of wound care coverings shall be described in the form of adhesive bandages having an adhesive strip and/or a pad mounted to the adhesive strip. However, embodiments shall also encompass standalone pads that can be applied as dressings for wounds in other manners, such as by being placed on an injured area and held in place by a gauze or fabric wrap, or by medical tape. The pads for wound care coverings in this disclosure may also be referred to as a carrier pad, a sheet, or a membrane.

In some embodiments, wound care coverings serve as an infection-alerting bandage, also referred to in this disclosure as an “alert-aid.” Wounds are known to accumulate lactate as a consequence of both anaerobic and aerobic glycolysis following microcirculation disruption, immune activation, and increased cell proliferation associated with wounds. Assessment of this lactate concentration in the fluid of the wound is helpful for confirming the suspicion of soft tissue infection, as higher levels of lactate indicate higher levels of cellular activity that are associated directly with a localized infection response. Embodiments of the present disclosure provide an easy-to-use and cost-effective product utilizing this rise in lactate concentration to provide a visual indicator to alert a user to a possible infection.

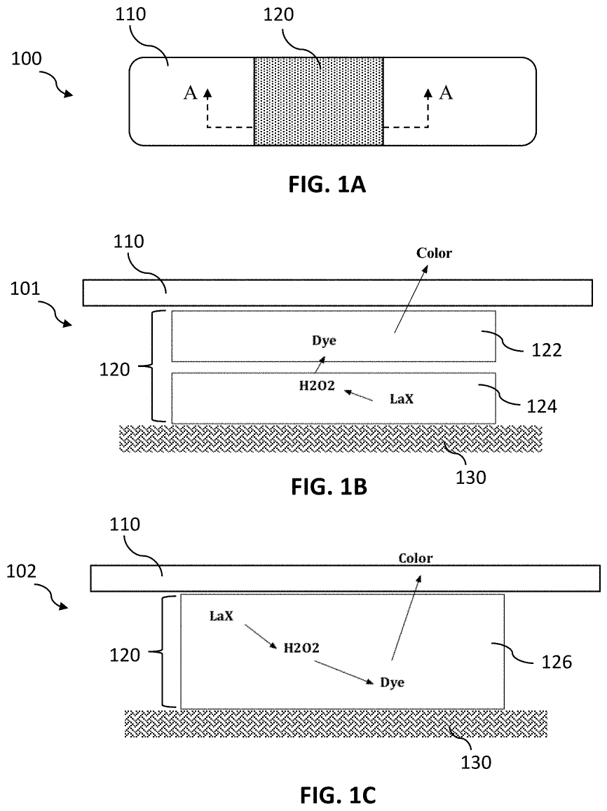

FIG. 1A shows a top view of an infection indication bandage 100 in which a color change occurs in the pad based on hydrogen peroxide concentration, accordance with some embodiments. FIGS. 1B and 1C show cross-sectional views of embodiments of the bandage pad portion (section A-A) of FIG. 1A. The figures are schematic and not drawn to scale.

In FIG. 1A, an adhesive strip 110 has a pad 120 mounted on it. The adhesive strip 110 is used to adhere the pad 120 on a user's skin 130. In some embodiments, the bandage 100 need not include the adhesive strip 110, but instead may comprise only the pad 120 to be placed on a user's wound manually and/or attached by other means (e.g., tape or a non-adhesive wrapping). The pad 120 may be mounted to (i.e., coupled to) the adhesive strip 110 by, for example, adhesive bonding, chemical bonding, or mechanical fastening (e.g., sewing or applying fasteners). The pad 120 may be gel-like in some embodiments and contains substances that cause a color change depending on the lactate levels that are detected in the wound that the pad is in contact with.

Various enzymes (e.g., lactate oxidase, lactate dehydrogenase) exist that convert lactate to a measurable quantity by conversion of lactate to hydrogen peroxide (H₂O₂) through a direct chemical reaction with the lactate. This reaction product concentration of hydrogen peroxide can result in a color change (e.g., from colorless to dark blue or purple) through reaction of hydrogen peroxide with a dye. The pad 120 of bandage 100 contains this type of dye that changes color in the presence of hydrogen peroxide, and also contains a lactate-based enzyme. The enzyme in the pad reacts with lactate in the wound to produce hydrogen peroxide, and the dye then indicates the presence of an infection via elevated lactate levels in the wound by changing color when H₂O₂ is detected. For example, the amount of enzyme and/or dye in the pad may be configured to trigger a color change when a certain threshold of lactate is detected (i.e., pad undergoes a color change when the threshold is reached or exceeded), where the threshold indicates a level high enough to indicate an infection.

In some embodiments, the dye is a colorimetric dye such as one or more of xylenol orange, titanium oxysulfate, titanium sulfate, 5,6-dimethyl phenanthroline (Fe complex), 2,2′-bipyridine (Fe complex), nitro phenanthroline (Fe complex), 1,10-phenanthroline iron(II) sulfate complex (Ferroin), and viologen. The color change can be detected by measuring a certain wavelength of color, a shift of the color, or alternatively by the intensity of the color. Observation of these changes may be accomplished by imaging the pad 120 with a camera on a smartphone (or other electronic device such as a computer tablet) and displaying results on an app in the phone as part of the wound care covering product (e.g., adhesive bandage 100). For example, existing peroxide assays measure absorbance of samples in the range of 550 nm to 650 nm, which can be used in conjunction with embodiments of the present disclosure.

In other embodiments, the dye is a fluorescing or chemiluminescent dye such as diphenyl oxalate, where the absence of presence of a color in the dye can be assessed visually by the user without a camera or imaging device. With fluorescing dyes, the presence of a color (e.g., changing from colorless to blue) indicates that lactate levels have risen above a certain threshold; a level high enough to represent infection.

The adhesive strip 110 (or other backing material/sheet) is transparent or translucent to allow a user to view the pad 120 through the adhesive sheet. For example, the adhesive strip 110 or backing material may be made of a transparent silicone or spun-lace acrylate.

The lactate-based enzymes are sensitive materials. The wound care covering embodiment of bandage 100 is uniquely constructed to provide encapsulation and stabilization of these materials in a fully biocompatible polymer carrier. This allows for both longevity (e.g., at least 5 days in-vivo use) and stabilization of the enzyme during sterilization of the product and also ensures that the enzymes remain in place in the pad. In embodiments, the pad comprises a pad material, and the lactate-based enzyme is crosslinked with the pad material by a crosslinker to provide this encapsulation and stabilization.

In some embodiments, the carrier pad material in pad 120 may be a hydrophilic polymer (e.g., hydrophilic polyurethane), a polymeric hydrogel or a natural hydrogel. Examples of polymeric hydrogels include poly(hydroxyethyl methacrylate) (i.e., poly-HEMA), polyethylene glycol (PEG), polyethylene glycol monoacrylate (PEGMA), cellulosics (e.g., carboxymethyl cellulose “CMC”), polyvinylpyrrolidone (PVP), and acrylamide. Examples of natural hydrogels include polysaccharides (e.g., alginate, chitosan), collagen, and fibrin. The enzymes can be crosslinked to the pad material by, for example glutaraldehyde, polyfunctional aziridine, bifunctional carbodiimide, dicyclohexyl carbodiimide, 1-ethyl-3-(3-dimethylaminopropyl)carbodiimide, N-hydroxysuccinimide, N-hydroxysulfosuccinimide, ethylene glycol bis(succinimidyl succinate) (EGS), ethylene glycol bis(sulfosuccinimidyl succinate) (SEGS), tris-(succinimidyl) aminotriacetate (TSAT), dimethyl pimelimidate (DMP), dimethyl suberimidate (DMS), 1,5-difluoro-2,4-dinitrobenzene (DFDNB), dimethyl 3,3′-dithiobispropionimidate (DTBP), NHS-Phosphine, NHS-PEG-azide, NHS-azide or combinations thereof.

The pad 120 may also contain initiators, cofactors, and/or mediators as required for the enzyme being used. For example, lactate dehydrogenase requires a mediator, which may be metallic-centered substances such as osmium or iron. Cofactors and/or initiators such as flavin adenin dinucleotide (FAD) may also be included to increase the speed of the enzyme reaction.

The carrier pad 120 can also hold the dye in close proximity to the enzyme such that when H₂O₂ is produced from the enzyme reaction, a concentration-dependent color signal is produced. FIG. 1B shows an embodiment of a bandage 101 (a cross-sectional view of section A-A of FIG. 1A) in which the dye and the lactate-based enzyme (“LaX”) are in separate layers of pad 120. The lactate-based enzyme is in an enzyme layer 124 of the pad, and the dye is in a dye layer 122 of the pad. In this embodiment, the enzyme layer 124 of the pad 120 is placed on the user's skin 130. The dye layer 122 is between the enzyme layer 124 and the adhesive strip 110. Lactate from the wound enters the enzyme layer 124 and reacts with the enzyme LaX, producing H₂O₂. The H₂O₂ enters the dye layer 122 of the pad 120, which is made of a hydrogel that contains the dye. The dye changes color (e.g., from colorless to purple or to blue) when H₂O₂ is present or is above a certain amount.

FIG. 1C shows another embodiment of a bandage 102 (a cross-sectional view of section A-A of FIG. 1A) in which both the dye and the lactate-based enzyme are located together in a layer 126 of the pad (i.e., dye and enzyme are combined in the same layer). In the embodiment of FIG. 1C, the enzymes are crosslinked with the carrier pad material as described above, and the dye is also dispersed throughout the pad 120.

In some embodiments, the color change can be evaluated on a binary basis (i.e., does the dye have a color or not). In other embodiments, the color change can be evaluated on a variable scale, where the specific color and/or intensity of the color can be used to indicate the amount of H₂O₂ present and thus the degree of infection. In certain embodiments, a smartphone application (or application on another type of mobile electronic device) can be used to scan the color change (e.g., intensity and hue) to determine whether there is an infection, rather than having the end user interpret degree of color change. For example, the optical density (i.e., absorbance) can be determined by scanning the pad with a smartphone camera, where the camera analyzes a specific wavelength or wavelength range associated with the dye reagent.

Embodiments of the bandages 100, 101 and 102 are configured as a wound care covering comprising a pad comprising a pad material; a lactate-based enzyme crosslinked with the pad material by a crosslinker; and a dye that changes color when hydrogen peroxide is present. The dye may be a colorimetric dye selected from xylenol orange, titanium oxysulfate, titanium sulfate, 5,6-dimethyl phenanthroline (Fe complex), 2,2′-bipyridine (Fe complex), nitro phenanthroline (Fe complex), 1,10-phenanthroline iron(II) sulfate complex (Ferroin), and viologen. The dye may be a chemiluminescent dye comprising diphenyl oxalate. The pad material may comprise a hydrophilic polymer, a polymeric hydrogel or a natural hydrogel. In some embodiments, the pad comprises an enzyme layer and a dye layer, wherein the enzyme layer comprises the lactate-based enzyme crosslinked with the pad material by the crosslinker, and the dye layer comprises the dye. In some embodiments, the pad has a layer that comprises both the lactate-based enzyme and the dye. In some embodiments the wound care covering further comprises an adhesive strip, wherein the pad is coupled to the adhesive strip. In further embodiments, the pad comprises an enzyme layer and a dye layer; the enzyme layer comprises the lactate-based enzyme; the dye layer comprises the dye; and the dye layer is between the enzyme layer and the adhesive strip.

Another use of hydrogen peroxide, besides being a reaction product that serves as an indicator of the presence of lactate, is a topical antiseptic used in wound cleaning that kills pathogens through oxidation burst and local oxygen production. H₂O₂ has been reported to be a reactive biochemical molecule synthesized by various cells like macrophages that influences biological behavior through multiple mechanisms—alterations of membrane potential, generation of new molecules, and changing intracellular redox balance—which results in activation or inactivation of different signaling transduction pathways. Contrary to the traditional viewpoint that H₂O₂ probably impairs tissue through its high oxidative property, scientific studies in the field have shown that a proper level of H₂O₂ is considered an important requirement for normal wound healing. (e.g., Loo et al., “Effects of Hydrogen Peroxide on Wound Healing in Mice in Relation to Oxidative Damage,” PLoS ONE 7(11): e49215, doi: 10.1371/journal.pone.0049215; and Zhu et al., “Hydrogen Peroxide: A Potential Wound Therapeutic Target?” Medical Principles and Practice 2017; 26: 301-308, doi: 10.1159/000475501.) Although conventional clinical use of H₂O₂ is still limited to the elimination of microbial contamination and sometimes hemostasis, low level production of H₂O₂ within wounds enhances the potential to exogenously augment and manipulate healing. Wound care coverings of the present disclosure utilize this finding to deliver hydrogen peroxide to an injury site at levels that promote healing.

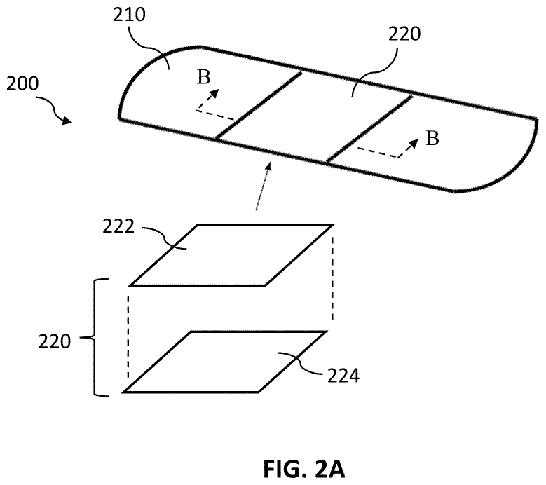

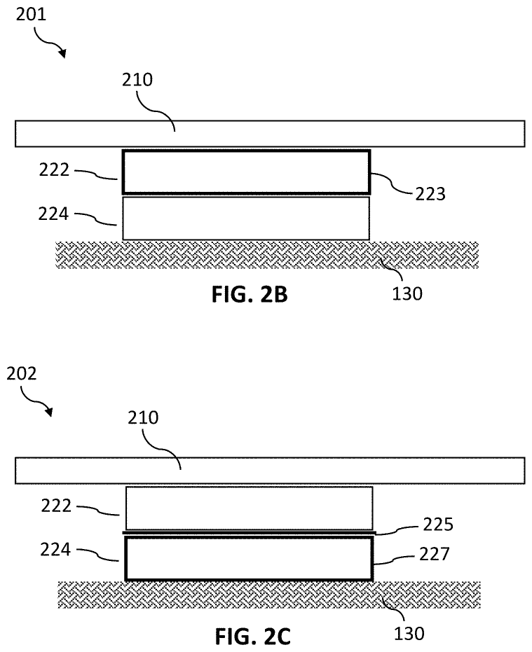

Some embodiments of the present disclosure include wound care coverings with therapeutic capabilities, which may also be referred to in this disclosure as a “prevent-aid.” FIG. 2A shows an exploded view of an infection-preventing bandage 200 that has an adhesive strip 210 and a pad 220. In some embodiments, the bandage 200 need not include the adhesive strip 210, but instead may comprise only the pad 220 to be placed on a user's wound. FIGS. 2B and 2C show cross-sectional views of section B-B in the pad area of FIG. 2A. The figures are schematic and not drawn to scale. The pad 220 in the bandage 200 has a sugar-containing layer 222 and an enzyme layer 224, where the sugar-containing layer 222 is between the enzyme layer 224 and the adhesive strip 210. That is, the enzyme layer 224 will contact the user's skin 130. The pad 220 functions by delivering hydrogen peroxide at a low level to the wound such that the hydrogen peroxide promotes healing.

Various enzymes exist that enable sugars to be assessed in a measurable quantity by conversion of sugar to hydrogen peroxide through a direct chemical reaction with the sugar. In the bandage 200, the concentration of the reaction product H₂O₂ is controlled to create very low continuous production of H₂O₂ over several days. The sugar in the sugar-containing layer 222 may be, for example, glucose, fructose, or galactose. For glucose as the sugar, the sugar-based enzyme may be glucose oxidase or glucose dehydrogenase. For fructose as the sugar, the sugar-based enzyme may be fructose dehydrogenase. For galactose as the sugar, the sugar-based enzyme may be galactose dehydrogenase. The sugar in the sugar-containing layer 222 may be contained in a hydrogel such as a silicone hydrogel.

In the bandage 200, the pad 220 uniquely provides encapsulation and stabilization of the sensitive sugar-based enzymes in a fully biocompatible polymer carrier. The enzyme layer 224 comprises a pad material and a sugar-based enzyme, where the sugar-based enzyme is crosslinked with the pad material by a crosslinker. The enzymes may be crosslinked to the pad material by, for example glutaraldehyde, polyfunctional aziridine, bifunctional carbodiimide, dicyclohexyl carbodiimide, 1-ethyl-3-(3-dimethylaminopropyl)carbodiimide, N-hydroxysuccinimide, N-hydroxysulfosuccinimide, EGS, SEGS, TSAT, DMP, DMS, DFDNB, DTBP, NHS-Phosphine, NHS-PEG-azide, NHS-azide or combinations thereof. This crosslinking allows for both longevity (e.g., at least 5 days in-vivo use) and stabilization of the enzyme during sterilization of the product and also ensures that the enzymes remain in place in the pad. In some embodiments, the carrier pad material may be a hydrophilic polymer (e.g., hydrophilic polyurethane), a polymeric hydrogel or a natural hydrogel. Examples of polymeric hydrogels include poly-HEMA, PEG, PEGMA, cellulosics (e.g., CMC), PVP, and acrylamide. Examples of natural hydrogels include polysaccharides (e.g., alginate, chitosan), collagen, and fibrin. Cofactors and initiators may be included in the enzyme layer as needed for the specific type of enzyme, and mediators may also be included (e.g., for dehydrogenase-type enzymes).

The polymeric carrier pad of the sugar-containing layer 222 releases the sugar into the enzyme layer 224 (e.g., glucose oxidase carrying membrane) in a controlled manner. This allows for slow production of hydrogen peroxide for several days and thus provides continuous production of H₂O₂ for wound cleaning and oxygen generation, thereby helping to prevent infection. The bandage 200 can be tuned to produce different levels of hydrogen peroxide based on controlling the flux of the sugars via a secondary enzyme encapsulation layer.

In some embodiments illustrated by bandage 201 in the cross-sectional view of FIG. 2B (section B-B of FIG. 2A), the sugar-containing layer 222 is constructed as a pouch, having the hydrogel material enclosed in a thin barrier 223 that can be ruptured by squeezing prior to use. In other embodiments illustrated by bandage 202 in the cross-sectional view of FIG. 2C (section B-B of FIG. 2A), a barrier 225 is included that is a sheet that is between the sugar-containing layer 222 and the enzyme layer 224. In either embodiment (FIG. 2B or 2C), the sugar-containing layer 222 contains a sugar solution in a flowable gel that permeates into the enzyme-containing layer 224. The pouch barrier 223 or sheet barrier 225 may be a polymer that can be easily ruptured by pressure applied by the user's fingers, such as being made of polyethylene terephthalate glycol (PETG) or cellulosics. In some embodiments, the rupturable barrier (pouch or sheet) may include perforations to facilitate rupturing of the barrier when pressure is applied by the user, and/or to promote flow of the sugar to the enzyme layer in particular locations along the pad.

The enzyme layer 224 contains a sugar-based enzyme crosslinked with a pad material as described above and may be encapsulated with a polymer or hydrogel. For example, the enzyme layer 224 may be covered with a hydrogel to ensure that the sugar gel (of the sugar-containing layer) remains in close contact with the enzyme layer 224. Additionally, the enzyme-containing pad 220 may be covered with a sugar limiting material 227 (e.g., glucose limiting material) as shown in FIG. 2C. That is, embodiments may include a sugar limiting material 227 surrounding the enzyme layer 224 to enable slow transfer of sugar from the sugar-containing layer 222 into the enzyme layer 224. The sugar limiting material 227 limits the rate of passage of sugar through the material, thus providing a titrating effect in the production of H₂O₂ from the enzyme layer 224. The sugar limiting material 227 (e.g., glucose limiting material) may be, for example, hydrophilic polyurethane, such as having a high molecular weight greater than 100,000 Daltons. The polyurethane of sugar limiting material 227 may be combined with polyacrylic acid, polyvinyl alcohol (PVA), polyvinylpyrrolidone (PVP) or poly(ethylene oxide) (PEO). In some embodiments, the sugar limiting material 227 may be a silicone combined with PEO.

Embodiments of the bandages 200, 201 and 202 include a wound care covering configured as an enzyme layer 224 comprising a pad material crosslinked with a sugar-based enzyme by a crosslinker; a sugar-containing layer 222 comprising a sugar in a hydrogel; and a rupturable barrier (e.g., barrier 223, barrier 225 and/or sugar limiting material 227) between the enzyme layer 224 and the sugar-containing layer 222.

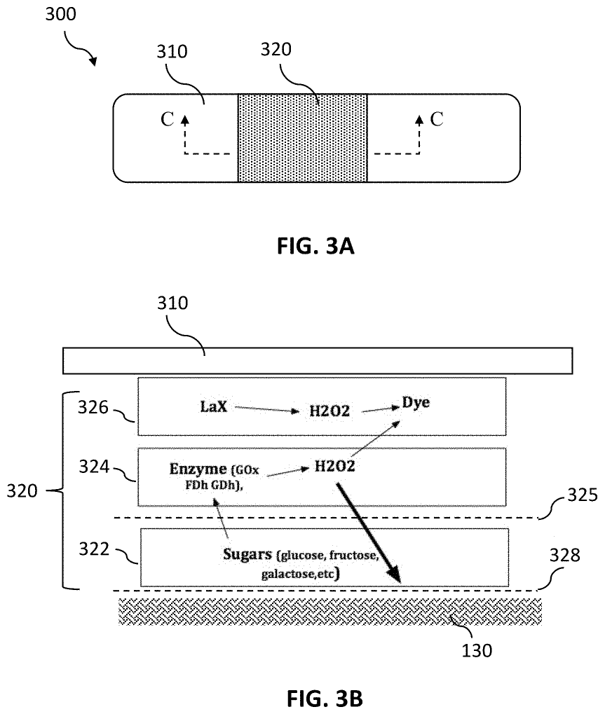

FIGS. 3A and 3B show an embodiment in which both the diagnostic (lactate detection via color change) and therapeutic (delivery of hydrogen peroxide) features are incorporated into a wound care covering bandage 300. FIG. 3A is a plan view, and FIG. 3B is a cross-sectional view of section C-C of the pad portion 320 of bandage 300. The figures are schematic and not drawn to scale. The adhesive strip 310 (or other backing material/sheet) is transparent or translucent to allow a user to view the pad 320 through the adhesive strip 310. For example, the adhesive strip 310 or backing material may be made of a transparent silicone or spun-lace acrylate. In some embodiments, the bandage 300 need not include the adhesive strip 310, but instead may comprise only the pad 320 to be placed on a user's wound.

As shown in FIG. 3B, pad 320 comprises a first enzyme layer 326 on the adhesive strip 310, a second enzyme layer 324 on the first enzyme layer 326, and a sugar-containing layer 322 on the second enzyme layer 324. The first enzyme layer 326 is a lactate-based enzyme (LaX) layer that produces a color change based on hydrogen peroxide concentration. The first enzyme layer 326 contains a lactate-based enzyme (e.g., lactate oxidase, lactate dehydrogenase) and a dye (e.g., colorimetric or fluorescing dye as described for bandage 100) that reacts with the hydrogen peroxide produced by the lactate reaction. The first enzyme layer 326 is similar to layer 126 of FIG. 1C in which the dye and lactate enzyme are combined in one layer. In embodiments, the first enzyme layer 326 includes a first pad material, a lactate-based enzyme and a dye that changes color in the presence of hydrogen peroxide, where the lactate-based enzyme is crosslinked with the first pad material by a first crosslinker. The first pad material and the first crosslinker may be materials as described in relation to FIGS. 1A-1C.

The second enzyme layer 324 is a sugar-based enzyme layer containing one or more sugar-based enzymes (glucose oxidase “GOx”; fructose dehydrogenase “FDh”; glucose dehydrogenase “GDh” are shown in the figure as examples). Second enzyme layer 324 is similar to enzyme layer 224 of FIGS. 2B-2C. In embodiments, the second enzyme layer 324 includes a second pad material and a sugar-based enzyme, where the second pad material is crosslinked with the sugar-based enzyme by a second crosslinker (e.g., as described in relation to FIGS. 2A-2C). The first pad material and the first crosslinker of first enzyme layer 326 may be the same or different than the second pad material and the second crosslinker of second enzyme layer 324.

The sugar-containing layer 322 includes a sugar in a hydrogel, where the second enzyme layer 324 is between the sugar-containing layer 322 and the first enzyme layer 326. The sugars (e.g., one or more of glucose, fructose, galactose) react with sugar-based enzymes delivered from the second enzyme layer 324 to produce hydrogen peroxide. The sugar-containing layer 322 provides titrated hydrogen peroxide delivery to the injury site for prevention of infection. A rupturable barrier 325 (as described for barrier 225 in relation to bandage 202) is between the second enzyme layer 324 and the sugar-containing layer 322 to help with titrating the production of H₂O₂ in the sugar/enzyme reaction. The rupturable barrier 325 may also help reduce inadvertent effects of H₂O₂ production from the sugar-containing layer 322 on the dye color change in the dye layer (first enzyme layer 326).

In FIG. 3B, the sugar-containing layer 322 is next to the skin 130 rather than the enzyme layer 224 being next to the skin as was the case for FIGS. 2B and 2C. In this manner, the sugar-containing layer 322 has access to the wound as well as to the second enzyme layer 324. In some embodiments, a skin barrier 328 such as a mildly permeable silicone or polyurethane layer may optionally be included next to the user's skin 130, over the sugar-containing layer 322, to limit the sugar from being absorbed into the body. For example, as shown in FIG. 3B, the skin barrier 328 is over the sugar-containing layer 322, opposite of a surface facing the second enzyme layer 324.

In some embodiments, the first enzyme layer 326, the second enzyme layer, 324 the sugar-containing layer 322, and the rupturable barrier 325 form a pad 320 that is coupled to the adhesive strip 310. In some embodiments, the first enzyme layer 326 is adjacent to the adhesive strip 310, the second enzyme layer 324 is on the first enzyme layer 326, and the sugar-containing layer 322 is on the second enzyme layer 324.

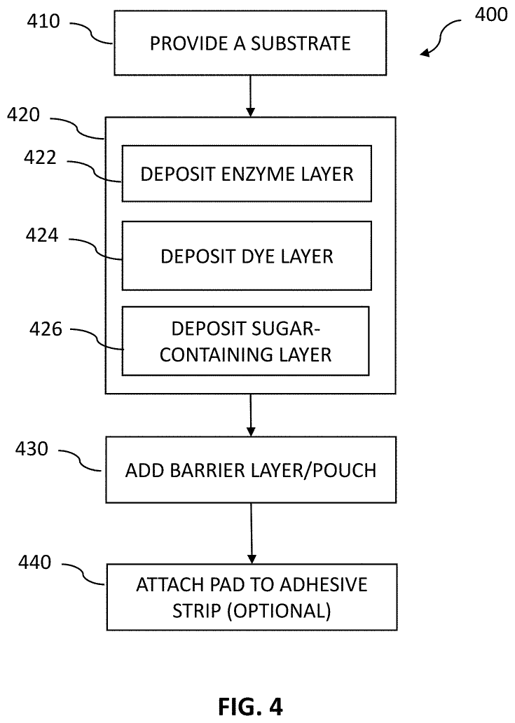

FIG. 4 is a flowchart 400 representing methods for producing the diagnostic and/or therapeutic wound care coverings of the present disclosure. In block 410, a substrate is provided, on which the pad material layers will be created. The substrate may be, for example, polyethylene terephthalate (PET), biaxially oriented polypropylene (BOPP), polyethylene (PE), nylon or acrylic polymers or even paper used in the industry for creating coating layers via a roller or web coating process. The substrate may be a sheet in the form a roll, to allow continuous production of the layers. In block 420, the layers of the pad are deposited. The layers may be deposited in any order as needed for the product being created. In block 422, the enzyme layer is deposited. The enzyme layer can be a lactate-based enzyme layer as described for FIGS. 1B-1C, a sugar-based enzyme layer as described for FIGS. 2B-2C, or can be two layers (both a lactate-based enzyme layer and a sugar-based enzyme layer) as described for FIG. 3B. In block 424, the dye layer is deposited for embodiments in which an infection-detecting wound care covering is being made. The dye layer may be a separate layer from the lactate-based enzyme layer of block 422 or may be combined with the lactate-based enzyme layer. In block 426, the sugar-containing layer is deposited for embodiments in which an infection-preventing wound care covering is being made. After all the pad layers have been created, the substrate having the pad material layers on it may then be cut into pads of a desired size. In block 430, barrier layer(s) and/or a pouch may be added. For example, a rupturable barrier pouch for the sugar-containing layer may be added by enclosing the sugar-containing layer with the pouch material, and heat sealing the edges. Block 430 may also include adding the barrier layers described in FIGS. 2C and 3B. Block 430 may be performed during block 420, such as to form a barrier around a particular layer before other layers are placed over that layer. In block 440, the pads (e.g., pads 120, 220, 320) may be coupled (e.g., adhered) to an adhesive strip (e.g., adhesive strips 110, 210, 310), for embodiments in which the wound care coverings are to be used as an adhesive bandage.

In an embodiment of making an infection-detecting wound care covering, block 422 of depositing the enzyme layer may be performed prior to block 424 of depositing the dye layer onto the enzyme layer, or vice versa, and block 426 is omitted. In another embodiment of making an infection-detecting wound care covering, block 422 and block 424 are combined, such that the lactate-based enzyme and dye are in the same layer of the pad. In an embodiment of making infection-preventing wound care covering, block 422 of depositing the enzyme layer may be performed prior to block 426 of depositing the sugar-containing layer onto the enzyme layer, or vice versa, and block 424 is omitted. In an embodiment of making a combination infection-detecting and infection-preventing wound care covering, block 424 (dye layer, which in this embodiment will also include the lactate-based enzyme) may be performed first, then block 422 to deposit the enzyme layer (sugar-based enzyme in this embodiment) on the dye layer, then block 426 to deposit the sugar-containing layer. Alternatively, the order may be reversed to start with block 426, then block 422 then block 424.

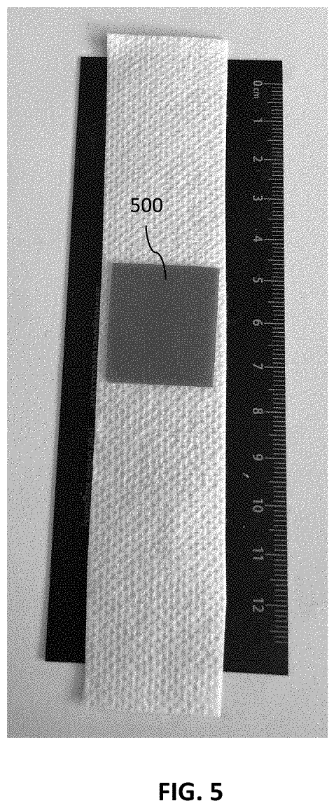

Prototype wound care pads were fabricated and tested in accordance with embodiments. FIG. 5 is a photograph of an example pad 500 comprising an enzyme layer to help prevent infection (e.g., per the embodiments of FIGS. 2A-2C and 3A-3B). The pad 500 may be used with or without a glucose packet (i.e., sugar-containing layer). Dimensions of the pad 500 were approximately 25 mm wide, 25 mm long, and 0.5 mm thick (approximately 1″×1″×0.020″). The pad 500 was fabricated using a lactate-based enzyme and a PEG-based hydrophile blended in an aliphatic, polyurethane dispersion and crosslinked with glutaraldehyde. The blend was covered in a silicone hydrogel layer.

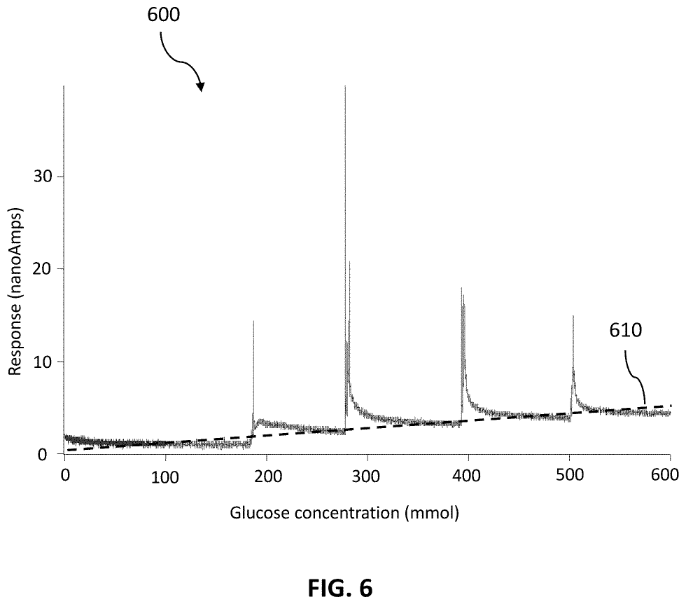

FIG. 6 is a graph 600 of the hydrogen peroxide response for the pad 500. The Y-axis of graph 600 is a measured electrical current (in nanoAmps) that indicates an amount of hydrogen peroxide produced when the pad was exposed to a series of stepwise increases in glucose solution (X-axis, in mmol). Measurements were made using a Digi-Ivy potentiostat and a platinum (Pt) wire to measure hydrogen peroxide directly. The data was collected by laying the Pt wire on the enzyme pad 500 (wetted with the glucose solution but not submerged) and measuring the hydrogen peroxide production of the enzyme pad 500 after exposure to different levels of glucose placed dropwise on the enzyme pad 500. The range shows a linear hydrogen peroxide response in the range of 2000-3500 pA/mmol, represented by the slope of line 610. These experimental results demonstrate that the membrane sheet (enzyme pad 500) can produce significant amounts of hydrogen peroxide in response to glucose, where the hydrogen peroxide can then be used for therapeutic purposes. Wound care coverings of the present disclosure can expose an enzyme layer to sugar (e.g., from a sugar-containing layer) when the covering is applied to a wound, resulting in the production of hydrogen peroxide which can help prevent infection in the wound.

As described herein, the wound care coverings of the present disclosure provide beneficial capabilities for detecting and preventing infections in wounds, in an easy-to-use and cost-effective manner.

Reference has been made in detail to embodiments of the disclosed invention, one or more examples of which have been illustrated in the accompanying figures. Each example has been provided by way of explanation of the present technology, not as a limitation of the present technology. In fact, while the specification has been described in detail with respect to specific embodiments of the invention, it will be appreciated that those skilled in the art, upon attaining an understanding of the foregoing, may readily conceive of alterations to, variations of, and equivalents to these embodiments. For instance, features illustrated or described as part of one embodiment may be used with another embodiment to yield a still further embodiment. Thus, it is intended that the present subject matter covers all such modifications and variations within the scope of the appended claims and their equivalents. These and other modifications and variations to the present invention may be practiced by those of ordinary skill in the art, without departing from the scope of the present invention, which is more particularly set forth in the appended claims. Furthermore, those of ordinary skill in the art will appreciate that the foregoing description is by way of example only and is not intended to limit the invention.

1. A wound care covering, comprising:
an enzyme layer comprising a pad material crosslinked with a sugar-based enzyme by a crosslinker;
a sugar-containing layer comprising a sugar in a hydrogel; and
a rupturable barrier between the enzyme layer and the sugar-containing layer.

2. The wound care covering of claim 1 wherein the pad material comprises a hydrophilic polymer, a polymeric hydrogel or a natural hydrogel.

3. The wound care covering of claim 1 wherein the hydrogel of the sugar-containing layer is a silicone hydrogel.

4. The wound care covering of claim 1 wherein the sugar is glucose, and the sugar-based enzyme is glucose oxidase or glucose dehydrogenase.

5. The wound care covering of claim 1 wherein the sugar is fructose, and the sugar-based enzyme is fructose dehydrogenase.

6. The wound care covering of claim 1 wherein the sugar is galactose, and the sugar-based enzyme is galactose dehydrogenase.

7. The wound care covering of claim 1 wherein the rupturable barrier comprises polyethylene terephthalate glycol (PETG).

8. The wound care covering of claim 1 wherein the rupturable barrier is a pouch surrounding the sugar-containing layer.

9. The wound care covering of claim 1 further comprising a sugar limiting material surrounding the enzyme layer.

10. The wound care covering of claim 9 wherein the sugar limiting material comprises a hydrophilic polyurethane having a molecular weight greater than 100,000 Daltons.

11. The wound care covering of claim 1 further comprising an adhesive strip;
wherein the enzyme layer, the sugar-containing layer and the rupturable barrier form a pad that is coupled to the adhesive strip.

12. The wound care covering of claim 11 wherein the sugar-containing layer is between the enzyme layer and the adhesive strip.

13.-20. (canceled)

21. A wound care covering, comprising:
a first enzyme layer comprising i) a first pad material, ii) a lactate-based enzyme and iii) a dye that changes color when hydrogen peroxide is present, wherein the lactate-based enzyme is crosslinked with the first pad material by a first crosslinker;
a second enzyme layer comprising a second pad material crosslinked with a sugar-based enzyme by a second crosslinker;
a sugar-containing layer comprising a sugar in a hydrogel, wherein the second enzyme layer is between the sugar-containing layer and the first enzyme layer; and
a rupturable barrier between the second enzyme layer and the sugar-containing layer.

22. The wound care covering of claim 21 wherein the dye is a colorimetric dye selected from xylenol orange, titanium oxysulfate, titanium sulfate, 5,6-dimethyl phenanthroline (Fe complex), 2,2′ -bipyridine (Fe complex), nitro phenanthroline (Fe complex), 1,10-phenanthroline iron(II) sulfate complex (Ferroin), and viologen.

23. The wound care covering of claim 21 wherein the dye is a chemiluminescent dye comprising diphenyl oxalate.

24. The wound care covering of claim 21 wherein the first pad material and the second pad material comprise a hydrophilic polymer, a polymeric hydrogel or a natural hydrogel.

25. The wound care covering of claim 21 further comprising an adhesive strip, wherein the first enzyme layer, the second enzyme layer, the sugar-containing layer, and the rupturable barrier form a pad that is coupled to the adhesive strip.

26. The wound care covering of claim 25 wherein the first enzyme layer is adjacent to the adhesive strip, the second enzyme layer is on the first enzyme layer, and the sugar-containing layer is on the second enzyme layer.

27. The wound care covering of claim 21 further comprising a skin barrier over the sugar-containing layer, opposite of a surface facing the second enzyme layer.
